(19) United States
(12) Patent Application Publication
(10) Pub. No.: US 2007/0259046 A1
(43) Pub. Date: Nov. 8, 2007

(54) Process for removal of solvents from bead polymers

(76) Inventors: Pierre Vanhoorne, Monheim (DE); Rheinhold Klipper, Koln (DE); Rudolf Wagner, Koln (DE); Wolfgang Podszun, Munchen (DE); Thomas Linn, Grevenbroich (DE)

Correspondence Address: LANXESS CORPORATION, 111 RIDC PARK WEST DRIVE, PITTSBURGH, PA, 15275-1112, US

(21) Appl. No.: 11/796,769

(22) Filed: Apr. 30, 2007

(30) Foreign Application Priority Data
May 5, 2006 (DE) 10 2006 020 874.9

(51) Int. Cl. C08F 6/00; A61K 38/28; A61K 9/14

(52) U.S. Cl. 424/489; 528/501; 514/3

(57) ABSTRACT

Process for removal of organic solvents from bead polymers, where the bead polymers are subjected to distillation of the organic solvent with addition of a water-soluble carboxylic acid.

The invention relates to a process for removal of organic solvent from bead polymers, where the bead polymers are subjected to distillation of the organic solvent with addition of a water-soluble carboxylic acid. The invention further relates to bead polymers whose content of organic solvents is less than 500 ppm, and also to their use for preparation of ion exchangers. The invention further relates to the use of hydrophilic carboxylic acids for removal of solvents from bead polymers.

Bead polymers are well known and by way of example are used for preparation of ion exchangers. The preparation of bead polymers is likewise known and by way of example can take place by way of bead polymerization of vinylic monomers. If the bead polymerization reaction is carried out in the presence of organic solvents (porogens), the product is known as porous bead polymers, these having a pore structure.

### BACKGROUND ART

As is known to the person skilled in the art, the pore structure can be controlled by varying content of crosslinking agent, and also by the nature and amount of the porogen. Porogens usually used are organic solvents, such as 4-methyl-2-pentanol, isobutanol, methyl isobutyl ketone, toluene, isooctane or isododecane, see for example U.S. Pat. No. 4,382,124, U.S. Pat. No. 3,586,646 and DE 1113570B1.

After the preparation process, the porogen generally has to be removed in order to free the pore structure. In some applications, e.g. the use of the porous bead polymers in the food-and-drink industry and in the pharmaceutical industry, the residual content of porogen is not permitted to exceed very low maximum values, typically a few ppm, for reasons related to toxicology.

There are also other organic solvents which have to be almost completely removed for the abovementioned applications, examples being residual amounts of unreacted monomers and of organic solvents which are used during the functionalization of porous and of non-porous bead polymers to give ion exchangers, to give substrate resins and to give functional adsorbers.

Various processes are used for removal of organic solvents from bead polymers.

In azeotropic distillation, the organic solvent is removed from an aqueous suspension of the bead polymers simply by distillation. The process functions only if the organic solvent has a certain degree of water-solubility, and it does not generally permit compliance with the low residual values required for the abovementioned applications.

As an alternative, the organic solvent can be extracted by other organic solvents. If the extractant is not water-soluble, it in turn has to be removed from the beads in an additional step. If it is water-soluble, it can be removed by washing the beads with water. However, the product here is large amounts of contaminated wastewater which have to be removed and lead to increased process costs. Furthermore, there is no guarantee here that low residual contents of organic solvents can be achieved.

Organic solvents can also be removed via steam-treatment of the bead polymers. However, the use of steam poses technical problems because the steam condenses in the bead polymer bed, and a very time-consuming operation is needed to achieve homogeneous treatment of all of the beads. Furthermore, the intensive thermal stress can collapse the pore structure of the beads in porous bead polymers if the softening point of the polymer network is exceeded.

If a volatile, low-boiling organic solvent has been used, drying of the beads can also lead to removal of the organic solvent. However, long drying times are needed in order to achieve low residual contents of organic solvents and large volumes of solvent-laden exhaust air are generated, and these have to be cleaned.

EP 1 350 809 A1 describes a process in which the organic solvent is removed by distillation with addition of water-soluble organic solvents (ethylene glycol derivatives). However, the distillation temperature in this process must be above the boiling point of the organic solvent to be removed, the result being increased energy consumption and possibly damage to the bead polymers, in particular if they have been functionalized.

This process does not ensure achievement of residual levels of organic solvents below 500 ppm. It is therefore an object of the present invention to provide a non-aggressive, low-cost, environmentally compatible process for removal of organic solvents from bead polymers.

The intention is that bead polymers with contents below 500 ppm of organic solvents be obtainable by way of the inventive process. Further objects of the invention are apparent from the description and from the examples.

### DISCLOSURE OF THE INVENTION

This object is achieved by a process for removal of organic solvents from bead polymers in which the bead polymers are subjected to distillation of the organic solvent with addition of a water-soluble carboxylic acid.

The invention further provides a bead polymer whose content of organic solvents is below 500 ppm. The invention further provides the use of bead polymers whose content of organic solvents is less than 500 ppm for preparation of ion exchangers. The invention further provides the use of hydrophilic carboxylic acids for removal of organic solvents from bead polymers.

For the purposes of the invention, organic solvents can be saturated, unsaturated and/or aromatic and they have from 1 to 30 carbon atoms, preferably from 1 to 20 carbon atoms, particularly preferably from 1 to 10 carbon atoms and, if appropriate, one or more heteroatoms.

Organic solvents which can be removed particularly effectively by the process of the present invention have water-solubility at 20° C. of less than 20% by weight, preferably less than 10% by weight and in particular less than 5% by weight. According to one particularly preferred embodiment of the invention, organic solvents are removed which are liquid at a temperature of from 0° C. to 100° C., preferably from 10 to 50° C. It is particularly advantageous that the boiling point of the organic solvents is below 220° C., preferably below 180° C., particularly preferably below 150° C. at 1 bar. By way of non-restrictive example, mention may be made of linear and branched alkanes containing from 5 to 12 carbon atoms, e.g. isododecane, isooctane, octane, heptane, hexane, pentane; cycloalkanes having from 5 to 12 carbon atoms, e.g. cyclohexane and methylcyclohexane; aromatic solvents, e.g. benzene, toluene, styrene, methylstyrene, α-methylstyrene, ethylstyrene, ethylbenzene, chlorostyrene, chloromethylstyrene and naphthalene; alcohols not water-soluble and having from 4 to 15 carbon atoms, e.g. n-butanol, 2-ethyl-1-hexanol and 4-methyl-2-pentanol; esters not water-soluble, e.g. ethyl acetate, n-butyl acetate, methyl acrylate, methyl methacrylate, n-butyl acrylate, n-butyl methacrylate, dioctyl phthalate and dibutyl phthalate; aliphatic ethers, e.g. diethyl ether and dibutyl ether; ketones not water-soluble, e.g. methyl isobutyl ketone and cyclohexanone; chlorinated alkanes, e.g. chloroform, dichloromethane, 1,2-dichloroethane and 1,2-dichloropropane; and also volatile silicones, e.g. hexamethyldisiloxane and decamethyltetrasiloxane. The inventive process can, of course, also remove mixtures of the abovementioned solvents.

The structure and preparation of bead polymers are known to the person skilled in the art. Bead polymers are usually solid, round particles whose average diameter is from 0.01 μm to 2000 μm. A preferred preparation process comprises the use of one or more vinylic monomers. By way of non-restrictive example, mention may be made of the following vinylic monomers: vinylaromatic monomers, such as styrene, methylstyrene, ethylstyrene, divinylbenzene, trivinylbenzene, vinylnaphthalene; acrylic and methacrylic monomers, such as methyl acrylate, ethyl acrylate, n-butyl acrylate, tert-butyl acrylate, 2-ethylhexyl acrylate, ethylene glycol diacrylate, methyl methacrylate, ethyl methacrylate, n-butyl methacrylate, tert-butyl methacrylate, 2-ethylhexyl methacrylate, 2-hydroxyethyl methacrylate, ethylene glycol dimethacrylate, acrylic acid, methacrylic acid, acrylonitrile and methacrylonitrile; vinyl ethers, such as ethyl vinyl ether, butyl vinyl ether, ethylene glycol divinyl ether, butanediol divinyl ether and diethylene glycol divinyl ether; vinyl esters, such as vinyl acetate and vinyl propionate and vinylsilanes, such as trimethoxyvinylsilane and triethoxyvinylsilane, and mixtures of the abovementioned compounds.

The inventive process is particularly suitable for removal of organic solvents from porous bead polymers. In the present invention, porous bead polymers are bead polymers whose pore volume, comprising micro-, meso- and macropores, is at least 0.1 cm³/g, preferably at least 0.25 cm³/g, determined by the test method suitable for the respective type of pore (nitrogen adsorption porosimetry for micro- and mesopores, mercury intrusion porosimetry for meso- and macropores). The terms micro-, meso- and macroporous have been previously described in detail in the technical literature (see, for example, Seidl et al. Adv. Polym. Sci., Vol. 5 (1967), pp. 113-213).

The distillation of the organic solvent to be removed is preferably carried out in an aqueous dispersion of the bead polymers. The solids content of the dispersion at the start of the distillation process is advantageously from 5% by weight to 50% by weight, but other solids contents are also possible. It is advantageous that the dispersion remains stirrable.

In order to render the dispersion temperature and distillation procedure homogeneous, the dispersion can be stirred during distillation. The stirrer rotation rate is advantageously set as a function of the size of the container and the nature of the stirrer in such a way as to give good mixing of the reactor contents without mechanical damage to the bead polymers. Stirrer rotation rates of from 10 to 250 rpm are typical.

According to one preferred embodiment of the invention, the distillation is carried out at 1 bar at the boiling point of the mixture present composed of water-soluble carboxylic acid, of organic solvent to be removed and, if appropriate, water. A particular feature of the process of the present invention is that there is absolutely no requirement that the boiling point of the water-soluble carboxylic acid added in the distillation process exceeds the boiling point of the solvent to be removed. By way of example, therefore, it is possible to remove toluene (boiling point 118° C. at 1 atm.) by distillation with addition of formic acid (boiling point 102° C. at 1 bar).

However, there is absolutely no restriction of the inventive process to distillation at a pressure of 1 bar. If necessary, the distillation can also, at reduced pressure, be carried out at temperatures lower than would be the case at 1 bar.

In the present invention, water-soluble carboxylic acids are those whose water-solubility at 20° C. is at least 5% by weight, preferably at least 10% by weight, particularly preferably at least 20% by weight. According to the invention, particularly suitable water-soluble carboxylic acids are those which are liquid at 20° C. and 1 bar. According to one preferred embodiment of the invention, water-soluble monocarboxylic acids are used as water-soluble carboxylic acids. Formic acid, acetic acid, propionic acid, butyric acid and isobutyric acid are particularly suitable. Formic acid, acetic acid and propionic acid are preferred, and formic acid is particularly preferred. Mixtures of the abovementioned acids can also be used.

The amount used of the water-soluble carboxylic acids is preferably at least 5% by weight, particularly preferably at least 10% by weight, and in particular at least 20% by weight. Their amount used is moreover preferably at most 90% by weight and particularly preferably at most 70% by weight, based in each case on the total amount of the aqueous phase. The concentration of water-soluble carboxylic acid is advantageously in the abovementioned concentration range during the course of the distillation for at least 1 h, preferably at least 2 h, particularly preferably at least 4 h.

The water-soluble carboxylic acid can be added in one or more portions. The water-soluble carboxylic acid is preferably added in one portion. Some of, or the entire amount of, water-soluble soluble carboxylic acid can also be used as initial charge immediately at the start of the distillation. The form in which the water-soluble carboxylic acid is added can be that of pure substance or of aqueous solution.

At the end of the inventive distillation procedure, the residual content of organic solvent in the bead polymers is preferably less than 500 ppm, particularly preferably less than 100 ppm, in particular less than 20 ppm and very particularly preferably less than 5 ppm, based in each case on the dry bead polymer.

The bead polymers prepared by the inventive process have excellent suitability for use as adsorber resins in the purification of gases and/or liquids, in particular of water and/or of aqueous solutions. The adsorber resins remove by way of example certain organic compounds selectively, e.g. chlorinated solvents, aromatic solvents, phenols and/or odorant substances from the liquids and/or gases mentioned.

Inventive bead polymers likewise have excellent suitability for use as means of separation in chromatography. They have particularly good suitability, if appropriate after functionalization, for the purification of solutions of biologically active compounds (active ingredients), such as those obtained inter alia in biotechnology from cell cultures and/or microorganisms specifically cultured for these purposes. Non-limiting examples which may be mentioned of these biologically active compounds are: antibiotics, e.g. cyclosporines, penicillins, tetracyclines, glycopeptides, macrolides, polypeptides, proteins, such as insulin, erythropoietin or coagulation factor VIII. Purification of solutions of biologically active compounds also includes the chromatographic purification of blood and/or blood plasma (known as dialysis) via selective adsorption, e.g. of cholesterol, toxins and/or immune complexes from blood.

The bead polymers prepared by the inventive process also have excellent suitability for the preparation of ion exchangers. They are advantageously converted into ion exchangers by the conventional functionalization methods known to the person skilled in the art.

For preparation of strongly acidic cation exchangers, the inventive bead polymers are preferably sulphonated. Suitable sulphonating agents in this case are sulphuric acid, sulphur trioxide and chlorosulphonic acid. Preference is given to sulphuric acid at a concentration of from 90 to 100%, particularly preferably from 96 to 99%. The sulphonation temperature is generally from 50 to 200° C., preferably from 90 to 130° C. If desired, a swelling agent can be used during sulphonation, examples being chlorobenzene, dichloroethane, dichloropropane or methylene chloride. After sulphonation, the reaction mixture composed of sulphonation product and residual acid is cooled to room temperature and diluted first with sulphuric acids of decreasing concentration and then with water. If desired, the inventively obtained cation exchanger in the H form can be treated for purification with deionized water at temperatures of from 70 to 145° C., preferably from 105 to 130° C. For many applications it is advantageous to convert the cation exchanger from the acidic form to the sodium form. Sodium hydroxide solution whose concentration is from 10 to 60%, preferably from 40 to 50%, is used for this conversion. The conversion temperature is also important. It has been found that conversion temperatures of from 60 to 120° C., preferably from 75 to 100° C., produce no defects in the ion exchanger beads and give particularly advantageous purity.

The inventive bead polymers can also be used for the preparation of anion exchangers. In this case, a suitable method is haloalkylation of the bead polymer with subsequent amination. A preferred haloalkylating agent is chloromethyl methyl ether. Reaction with a secondary amine, such as dimethylamine, can give weakly basic anion exchangers from the haloalkylated polymers. Correspondingly, reaction of the haloalkylated polymers with tertiary amines, such as trimethylamine, dimethylisopropylamine or dimethylaminoethanol, gives strongly basic anion exchangers.

The process known as the phthalimide process can also be used to prepare anion exchangers via amidoalkylation of the bead polymer. For preparation of the amidomethylating reagent, by way of example, phthalimide or a phthalimide derivative is dissolved in a solvent and formalin is admixed. A bis(phthalimido) ether is then formed therefrom with elimination of water. The bis(phthalimido) ether can, if appropriate, be converted to the phthalimido ester. Preferred phthalimide derivatives for the purposes of the present invention are phthalimide itself or substituted phthalimides, such as methyl phthalimide. Solvents used in the preparation of the amidomethylating reagent are inert solvents suitable for swelling the polymer, preferably chlorinated hydrocarbons, particularly preferably dichloroethane or methylene chloride. For functionalization, the crosslinked bead polymer from step c) of the process is reacted with the amidomethylating agent. The catalyst used here comprises oleum, sulphuric acid or sulphur trioxide. The reaction temperature here is from 20 to 120° C., preferably from 50 to 100° C. The cleavage of the phthalic acid radical and thus the release of the aminomethyl group is achieved by treatment of the amidomethylated crosslinked bead polymer with aqueous or alcoholic solutions of an alkali metal hydroxide, such as sodium hydroxide or potassium hydroxide, at temperatures of from 100 to 250° C., preferably from 120 to 190° C. The concentration of the sodium hydroxide solution is in the range from 10 to 50% by weight, preferably from 20 to 40% by weight. The aminomethylated bead polymer obtained is finally washed with deionized water until free from alkali. In a further step of the process, the bead polymer containing aminomethyl groups is converted to anion exchanger by reaction with alkylating agents. The alkylation preferably takes place by the Leuckart-Wallach method. A Leuckart-Wallach reagent with particularly good suitability is formaldehyde in combination with formic acid as reducing agent. The alkylation reaction is carried out at temperatures of from 20 to 150° C., preferably from 40 to 110° C., and at pressures of from atmospheric pressure to 6 bar. Following the alkylation, the weakly basic anion exchanger obtained can be quaternized entirely or to some extent. By way of example, the quaternization can take place with methyl chloride. EP-A 1 078 688 describes by way of example further details of the preparation of anion exchangers by the phthalimide process.

Chelating resins can also be readily prepared from the inventive bead polymers. For example, reaction of a haloalkylated polymer with iminodiacetic acid gives chelating resins of iminodiacetic acid type.

The section below uses some examples to illustrate the inventive process. These examples serve merely as a guide and constitute absolutely no restriction of the inventive process described above. It will be understood that the specification and examples are illustrative but not limitative of the

present invention and that other embodiments within the spirit and scope of the invention will suggest themselves to those skilled in the art.

### EXAMPLES

### Example 1

### Removal of Toluene via Simple Distillation (Non-Inventive)

Porogen-saturated bead polymer was prepared by suspension polymerization of a mixture composed of divinylbenzene (473 g, technical quality, 80% purity), toluene (720 g) and tert-butyl 2-ethylperoxohexanoate (2.8 g).

480 g of the bead polymer obtained were used as initial charge with 1500 ml of water in a 4-litre stirred reactor equipped with thermostat, stirrer and distillation bridge, and distillation was continued until the boiling point in the reactor reached 100° C. and the only remaining distillate produced was water. The distillate obtained was a two-phase mixture composed of 280 ml of toluene and 760 ml of water.

10 ml of bead polymer were removed and washed with water. The residual toluene content determined via gas chromatography was 9800 ppm (0.98%).

### Example 2

### Removal of Toluene with Addition of Formic Acid (Inventive)

500 ml of formic acid (85% strength in water) were added to the remaining bead polymer from Comparative Example 1 and distillation was continued until the amount of distillate in this step reached 750 ml. The temperature in the reactor rose as far as 104° C.

10 ml of bead polymer were removed and washed with water. The residual content of toluene in the bead polymer, determined by gas chromatography, was 200 ppm.

300 ml of water and a further 500 ml of formic acid (85% strength in water) were added to the remaining bead polymer and a further 750 ml of liquid were distilled.

The liquid in the reactor was removed by suction and replaced by 1500 ml of water which was again removed by suction. The beads were again mixed with 1500 ml of water and 45 g of sodium hydroxide at 80° C. for 2 h and then washed with water in a suction filtration funnel until the pH of the eluate was <8.

The residual content of toluene in the bead polymer, determined by gas chromatography, was smaller than 15 ppm. The residual content of sodium formate, determined by ion exchange chromatography, was smaller than 5 ppm.

### Example 3

### Removal of Toluene with Addition of Formic Acid (Inventive)

Porogen-saturated bead polymer was prepared by suspension polymerization of a mixture composed of divinylbenzene (256 kg, technical quality, 80% purity), toluene (384 kg) and tert-butyl 2-ethylperoxohexanoate (1.54 kg).

200 litres of the bead polymer obtained were used as initial charge together with 550 litres of formic acid (32% strength in water) in a 1000 litre reactor composed of HC steel, equipped with jacket heating and distillation column. Distillation was continued until 67 kg of toluene and 400 litres of aqueous phase had been condensed. The temperature in the reactor rose as far as 106° C.

500 ml of bead polymer were removed and washed with water. The residual content of toluene in the bead polymer, determined by gas chromatography, was 930 ppm.

25 kg of formic acid (from 98 to 100% strength) and 25 litres of water were added to the reactor contents and a further 50 litres of distillate were removed. 200 litres of water were then added and again 200 litres of distillate were removed.

The reactor contents were transferred to a suction filtration funnel and the bead polymer was washed twice with 187 litres of water, with 134 litres of sodium hydroxide solution (4% by weight) and 4 times with 200 litres of water on the filter.

The residual content of toluene in the bead polymer, determined by gas chromatography, was 190 ppm.

### Example 4

### Removal of MIBK by Simple Distillation (Non-Inventive)

Porogen-saturated bead polymer was prepared by suspension polymerization of a mixture composed of divinylbenzene (240 g, technical quality, 80% purity), styrene (80 g), methyl isobutyl ketone (MIBK, 480 g) and dibenzoyl peroxide (3.2 g, 75% purity).

624 g of the bead polymer obtained were used as initial charge with 1500 ml of water in a 4-litre stirred reactor equipped with thermostat, stirrer and distillation bridge, and distillation was continued until the boiling point in the reactor reached 100° C. and the only remaining distillate produced was water.

10 ml of bead polymer were removed and washed with water. The residual content of MIBK, determined by gas chromatography, was 2% (2000 ppm).

### Example 5

### Removal of MIBK with Addition of Formic Acid (Inventive)

500 ml of formic acid (85% strength in water) were added to the remaining bead polymer from Example 1 and distillation was continued until the amount of distillate in this step reached 750 ml. The temperature in the reactor rose as far as 104° C.

10 ml of bead polymer were removed and washed with water. The residual content of methyl isobutyl ketone (MIBK) in the bead polymer, determined by gas chromatography, was 75 ppm.

300 ml of water and a further 500 ml of formic acid (85% strength in water) were added to the remaining bead polymer and a further 750 ml of liquid were distilled.

The liquid in the reactor was removed by suction and replaced by 1500 ml of water which was again removed by suction. The beads were again mixed with 1500 ml of water and 45 g of sodium hydroxide at 80° C. for 2 h and then washed with water in a suction filtration funnel until the pH of the eluate was <8.

The residual content of MIBK in the bead polymer, determined by gas chromatography, was smaller than 15 ppm. The residual content of sodium formate, determined by ion exchange chromatography, was smaller than 7 ppm.

### Example 6

### Removal of DCE with Addition of Formic Acid (Inventive)

Monodisperse, porous bead polymer whose average bead diameter was 33 μm according to WO 2005/075530 was prepared from 725.6 g of monodisperse, non-crosslinked polystyrene seed polymer (average bead diameter 18 μm), 5442 g of divinylbenzene, 544 g of toluene and 181 g of tert-butyl 2-ethylperoxohexanoate.

The bead polymer was isolated and dried in vacuo at 80° C. for 24 h.

150 g of the dried bead polymer were extracted 3 times with 1500 ml of 1,2-dichloroethane (DCE) in a 4-litre reactor equipped with stirrer, thermostat and distillation bridge, in order to remove the soluble polystyrene deriving from the seed polymer.

At the end of the third extraction procedure, the DCE was removed by suction (while the lower density of the bead polymer caused them to float) and 1500 ml of water and 500 ml of formic acid (85% strength in water) were added. Distillation was continued until 140 ml of DCE and 1100 ml of aqueous phase had been condensed.

The residual content of 1,2-dichloroethane in the bead polymer, determined by gas chromatography, was smaller than 20 ppm.

Comparable results are also obtained when using the water-soluble carboxylic acids mentioned by way of alternative in the description.

### Example 7

### Removal of Isododecane with Addition of Propionic Acid (Inventive)

Porogen-saturated bead polymer was prepared by suspension polymerization of a mixture composed of styrene (872.2 g), divinylbenzene (65.1 g, technical quality, 80% purity), isododecane (technical quality, 591 g) and tert-butyl 2-ethylperoxohexanoate (5.73 g).

750 g of the bead polymer obtained were used as initial charge with 1500 ml of propionic acid (78% strength in water) in a 4-litre stirred reactor equipped with thermostat, stirrer and distillation bridge, and distillation was continued until the distillate produced was only water and comprised no isododecane.

500 ml of propionic acid (78% strength in water) were then added and distillation was continued until the amount of distillate in this step reached 500 ml.

10 ml of bead polymer were removed and washed with water. The residual content of isododecane in the bead polymer, determined by gas chromatography, was 200 ppm. A further 500 ml of propionic acid (78% strength in water) were added to the remaining bead polymer and a further 500 ml of liquid were distilled.

Once again 10 ml of bead polymer were removed and washed with water. The residual content of isododecane in the bead polymer, determined by gas chromatography, was 75 ppm. A further 500 ml of propionic acid (78% strength in water) were added to the remaining bead polymer and a further 500 ml of liquid were distilled.

The liquid in the reactor was removed by suction and replaced by 1500 ml of water which was again removed by suction. The beads were again mixed with 1500 ml of water and 45 g of sodium hydroxide at 80° C. for 2 h and then washed with water in a suction filtration funnel until the pH of the eluate was <8.

The residual content of isododecane in the bead polymer, determined by gas chromatography, was smaller than 20 ppm.

1. A bead polymer, comprising less than 500 ppm of residual content of organic solvents.

2. A process for removal of organic solvents from bead polymers, wherein the bead polymers are subjected to distillation of the organic solvent with addition of a water-soluble carboxylic acid.

3. A process according to claim 2 wherein during the course of the distillation the concentration of the water-soluble carboxylic acid in the aqueous phase is adjusted in such a way as to exceed 10% by weight.

4. A process according to claim 2 wherein the water-soluble carboxylic acid is added in a single portion during the course of the distillation.

5. A process according to claim 2 wherein at least some of the water-soluble carboxylic acid is used as initial charge immediately at the start of the distillation.

6. A process according to claim 2 wherein formic acid, acetic acid, propionic acid, butyric acid, isobutyric acid or a mixture thereof are used as water-soluble carboxylic acid.

7. A process according to claim 2 wherein the bead polymer is subjected to the distillation in an aqueous dispersion.

8. A process according to claim 2 wherein a monocarboxylic acid which is liquid at 20° C. and 1 atm is used as water-soluble carboxylic acid.

9. A method of using bead polymers obtained by claim 2 for preparation of ion exchangers or of adsorber resins.

10. Use of hydrophilic carboxylic acids for removal of hydrophobic solvents from bead polymers.

11. A method of using bead polymers according to claim 1 for purification of solutions of biologically active compounds.

12. A method of using bead polymers obtained by claim 11 wherein the biologically active compound is insulin.
